(19) United States
(12) Patent Application Publication
(10) Pub. No.: US 2022/0062083 A1
(43) Pub. Date: Mar. 3, 2022

(54) SUCTION DEVICE FOR SUCTIONING, AEROSOLS, SALIVA, BLOOD, SERUM AND OR ANY OTHER LIQUID FROM PROCEDURES OR SURGERIES DONE ON HUMANS OR ANIMALS

(71) Applicant: Robert Hazany, Canoga Park, CA (US)

(72) Inventor: Robert Hazany, Canoga Park, CA (US)

(21) Appl. No.: 17/461,845

(22) Filed: Aug. 30, 2021

Related U.S. Application Data
(60) Provisional application No. 63/071,743, filed on Aug. 28, 2020.

(51) Int. Cl. A61G 13/10; A61G 15/14; A61C 17/06

(52) U.S. Cl. CPC A61G 13/108; F24F 2221/36; A61C 17/06; A61G 15/14

(57) ABSTRACT

A suction device including a funnel including an opening at an end and a base at an opposite end, wherein a diameter of the opening is wider than a diameter of the base; a tube; and a suction motor, wherein a first end of the tube is coupled to the base of the funnel via a connector and a second end of the tube is coupled to the suction motor, and wherein the suction motor is configured to generate a suction force that pulls aerosols from surrounding environment into the opening of the funnel and through the tube and into a collection reservoir for disposal.

### CROSS-REFERENCE TO RELATED APPLICATION(S)

This application claims priority to and the benefit of U.S. Provisional Patent Application No. 63/071,743 filed Aug. 28, 2020 and entitled “A SUCTION DEVICE FOR SUCTIONING, AEROSOLS, SALIVA, BLOOD, SERUM, AND OR ANY OTHER LIQUID FROM PROCEDURES OR SURGERIES DONE ON HUMANS OR ANIMALS”, the entire content of which is incorporated herein by reference.

### BACKGROUND

### 1. Field

Some embodiments of the present disclosure relate generally to a suction device for suctioning aerosols, saliva, blood, serum, and or any other liquid from procedures or surgeries done on humans or animal.

### 2. Description of the Related Art

Various professions may be at greater risk of contracting an infectious disease due to regular exposure to aerosols (e.g., tiny particles or droplets suspended in the air, such as dusts, mists, or fumes) that are propelled from human oral and nasal cavities in the workplace. Many infectious diseases spread to other people by using aerosols as vehicles and becoming inhaled by other people or absorbed into their skin. Many of the professions may include workers in medical offices, surgical centers, hospitals, veterinary hospitals, or various other locations.

Preventive measures can be implemented to reduce the risk of contracting an infectious diseases by limiting the spread of aerosols. For example, workers in the workplace can wear a cloth covering over their oral and nasal cavities to prevent spreading of aerosols. However, some professions face unique challenges that can limit preventive measure options. For example, health care workers are regularly exposed to aerosols propelled from oral and nasal cavities of their patients. Health care workers can wear personal protective equipment (PPE) that can limit exposure to aerosols. However, some aerosols may not be fully prevented by PPE. Further, aerosols may remain in the air for extended periods of time, and can come into contact with workers after they have removed their PPE. Additional steps for capturing and removing aerosols from the air can be helpful in reducing the risks of contracting infectious diseases.

### SUMMARY

Aspects of embodiments of the present disclosure are directed toward a suction device for suctioning aerosols, saliva, blood, serum, and/or any other liquid from procedures or surgeries done on humans or animals.

According to some example embodiments of the present disclosure, a suction device including: a funnel including an opening at an end and a base at an opposite end, wherein a diameter of the opening is wider than a diameter of the base; a tube; and a suction motor, wherein a first end of the tube is coupled to the base of the funnel via a connector and a second end of the tube is coupled to the suction motor, and wherein the suction motor is configured to generate a suction force that pulls aerosols from surrounding environment into the opening of the funnel and through the tube and into a collection reservoir for disposal.

According to some example embodiments, the base of the funnel includes a flexible base.

According to some example embodiments, the suction motor includes a heating, ventilation, and air conditioning (HVAC) suction system.

According to some example embodiments, the diameter of the opening of the funnel has a range of 4 to 5 inches.

According to some example embodiments, the connector includes a plurality of support portions that stabilizes the connector.

According to some example embodiments, the tube includes an angled portion.

According to some example embodiments, the base of the funnel includes a rigid base.

According to some example embodiments of the present disclosure, a suction device including: a funnel including an opening at an end and a base at an opposite end, wherein a diameter of the opening is wider than a diameter of the base; a tube includes an upper portion section and a lower portion section, wherein the upper portion section is configured to be coupled and decoupled from the lower portion section; and a suction motor, wherein a top end of the upper portion section of the tube is connected to the base of the funnel via a connector and a bottom end of the lower portion section is coupled to the suction motor, wherein a bottom end of the upper portion section of the tube is coupled to a top end of the lower portion section of the tube, and wherein the suction motor is configured to generate a suction force that pulls aerosols from surrounding environment into the opening of the funnel and through the tube and into a collection reservoir for disposal.

According to some example embodiments, the base of the funnel includes a flexible base.

According to some example embodiments, the suction motor includes a heating, ventilation, and air conditioning (HVAC) suction system.

According to some example embodiments, the diameter of the opening of the funnel has a range of 4 to 5 inches.

According to some example embodiments the connector includes a plurality of support portions that stabilizes the connector.

According to some example embodiments, the tube includes an angled portion.

According to some example embodiments, the base of the funnel includes a rigid base.

According to some example embodiments of the present disclosure, in a method for removing aerosols expelled by oral and nasal cavities of a person using a suction device, the method including: coupling a funnel to a first end of a tube via a connector, wherein the funnel includes an opening at an end and a base at an opposite end, and wherein the base is coupled to the tube via the connector; coupling a second end of the tube to a suction motor; positioning the opening of the funnel at a distance from the oral and nasal cavities of the person; and activating the suction motor, wherein the suction motor is configured to generate a suction force that pulls aerosols from surrounding environment into an opening of the funnel.

According to some example embodiments, the base of the funnel includes a flexible base.

According to some example embodiments, the suction motor includes a heating, ventilation, and air conditioning (HVAC) suction system.

According to some example embodiments, wherein the connector includes a plurality of support portions that stabilizes the connector.

According to some example embodiments, the tube includes an angled portion.

According to some example embodiments, the base of the funnel includes a rigid base.

### BRIEF DESCRIPTION OF THE DRAWINGS

FIG. 1 is a front view of the suction device according to an embodiment of the present disclosure.

FIG. 2 is a side view of the suction device according to an embodiment of the present disclosure.

FIG. 3 is a second side view of the suction device according to an embodiment of the present disclosure.

FIG. 4 is a third side view of the suction device according to an embodiment of the present disclosure.

FIGS. 5-7 show another illustration of the suction device according to an embodiment of the present disclosure.

FIGS. 8-10 show another illustration of the suction device according to an embodiment of the present disclosure.

FIGS. 11 and 12 show another illustration of the suction device according to an embodiment of the present disclosure.

FIG. 13 illustrates a suction device according to an example embodiment of the present disclosure.

FIG. 14 illustrates two-part suction device according to an example embodiment of the present disclosure.

FIG. 15 illustrates a second two-part suction device according to an example embodiment of the present disclosure.

### DETAILED DESCRIPTION

The exemplary embodiments of the present invention disclosed herein are directed to a suction device for suctioning saliva in and adjacent to an oral cavity.

Features of the inventive concept and methods of accomplishing the same may be understood more readily by reference to the following detailed description of embodiments and the accompanying drawings. Hereinafter, embodiments will be described in more detail with reference to the accompanying drawings, in which like reference numbers refer to like elements throughout. The present disclosure, however, may be embodied in various different forms, and should not be construed as being limited to only the illustrated embodiments herein. Rather, these embodiments are provided as examples so that this disclosure will be thorough and complete, and will fully convey the aspects and features of the present disclosure to those skilled in the art. Accordingly, processes, elements, and techniques that are not necessary to those having ordinary skill in the art for a complete understanding of the aspects and features of the present disclosure may not be described. Unless otherwise noted, like reference numerals denote like elements throughout the attached drawings and the written description, and thus, descriptions thereof will not be repeated. The drawings are not necessarily to scale and the relative sizes of elements, layers, and regions shown may be exaggerated for clarity.

Embodiments of the present disclosure include a suction device (e.g., an aerosuction device) for suctioning aerosols in and adjacent to oral and nasal cavities. In some embodiments, the suction device can be used with various suction systems, including, but not limited to, an HVAC suction system used in medical, dental, and veterinary offices. In some embodiments, the suction device can be made with different materials and can be used for medical purposes in offices, surgical centers, hospitals, veterinary hospitals, and other locations. The suction device can reduce the presence of viruses and bacteria during procedures for human and animals by approximately 95%, which can help protect doctors and staff.

According to one embodiment, the device can include a tube connected to a funnel shaped portion (e.g., a cup) at one end and connected to a suction motor at the other end. In some embodiments, the cup may be made using various materials that are suitable for only a one-time use and disposable (e.g., plastic)). In other embodiments, the cup may be made using various materials that are suitable for repeated use and capable of being sterilized (e.g., metal). In various embodiments, the cup can have an opening at one end that can be positioned over the oral and nasal cavities of a person such that it can capture aerosols propelled from the cavities. In various embodiments, the suction device can also be connected to a suction motor that generates a suction force that can pull aerosols from the air into the opening of the cup. In some embodiments, the suction motor can be an HVAC suction system used in medical, dental, and veterinary offices. After being captured, the aerosols can travel through the tube to the collection reservoir for disposal.

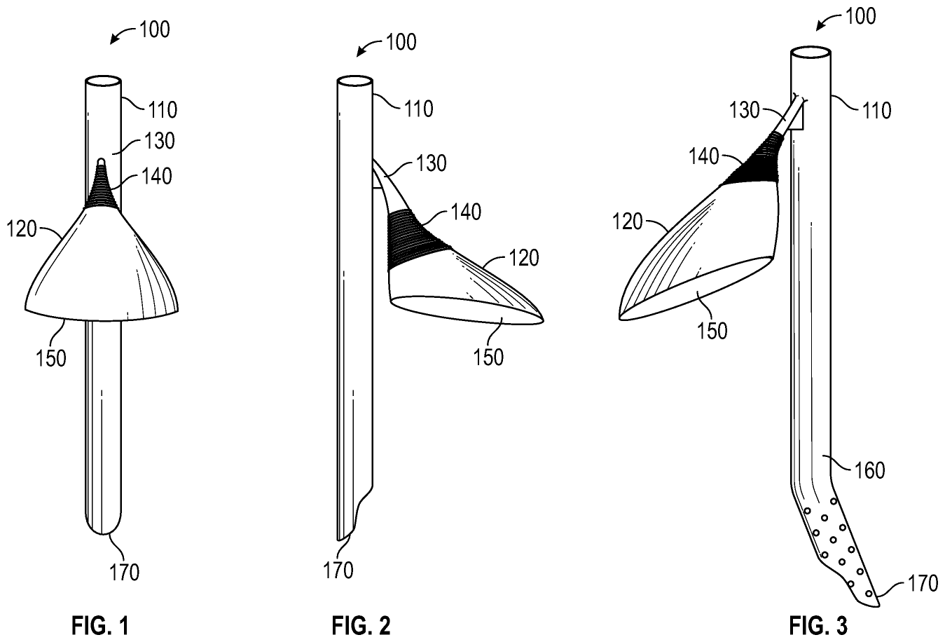

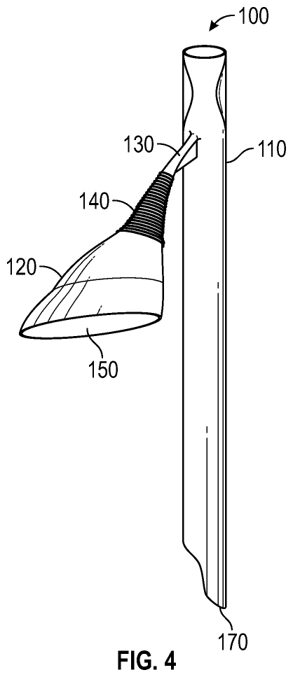

Referring to FIGS. 1-4, a suction device 100 according to an embodiment of the present invention may include a tube 110 connected to a cup 120 via a connector 130 at one end and connected to a suction motor at the opposite end 170. In some embodiments, the cup 120 can be a funnel shaped portion and can include a flexible base 140 at one end near or adjacent to the connector 130 and an opening 150 at the opposite end. In some embodiments, the diameter of the opening 150 can be wider than the diameter of the flexible base 140. The cup opening 150 can be positioned near the oral and nasal cavities of a person and capture aerosols propelled from the cavities and other aerosols from the surrounding environment. In some embodiments, the cup opening 150 can be positioned approximately 3 inches from a person's oral and nasal cavities. In some embodiments, the diameter of the opening 150 can be 4-5 inches. The suction device 100 can be connected to a suction motor at end 170. The suction motor can generate a suction force that can help pull the aerosols propelled from the oral and nasal cavities and from the surrounding environment into the cup 120. In some embodiments, the suction motor can be an HVAC suction system used in medical, dental, and veterinary offices. After the aerosols are captured by the cup 120, the aerosols can travel through the tube 110. Subsequently, the aerosols can be collected in a collection reservoir for disposal.

In some embodiments, the cup 120 can be rigidly connected (e.g., not being able to move or pivot) to the tube 110 via a connector 130. The connector 130 can include additional support elements (e.g., support portions) to help maintain a sturdy connection between the cup 120 and the tube 110. In some embodiments, the additional support portions may include rigid materials that extends from the tube 110 to the connector 130 or from the tube 110 to a base of the cup 120. In some embodiments, the cup 120 can include a flexible base 140 that is positioned adjacent to the connector 130. The flexible base 140 can include flexible material that can allow the cup 120 to be adjusted closer or further from the oral and nasal cavities of a person. Additionally, the flexible base 140 can allow the angle of the opening to be adjusted to different angles.

In some embodiments the tube 110 can be bent at an angle 160 as shown in FIG. 3. The angle 160 can allow the cup 120 to be positioned over the oral and nasal cavities of a person such that the tube 110 does not come into contact with the person. Additionally, the angle 160 can be positioned at various points along the length of the tube 110. In some embodiments, the angle 160 may range between 15 to 35 degrees. In some embodiments, the tube 110 may be made with a rigid material (e.g., not flexible), including, but not limited to, plastic. Also, according to various embodiments, the length of the tube 110 can vary. In one embodiment, the length of the tube 110 may range from 5 to 6 inches. In one embodiment, the tube 110 may range from 8 to 10 inches. The tube 110 can include an end 170 that is opposite the end connected to the cup 120. The end 170 can be connected to a suction motor that can generate a suction force that can pull the aerosols propelled from the oral and nasal cavities into the cup 120. In some embodiments, the suction motor can be an HVAC suction system used in medical, dental, and veterinary offices. After the aerosols are captured by the cup 120, the aerosols can travel through the tube 110, and into a collection reservoir for disposal.

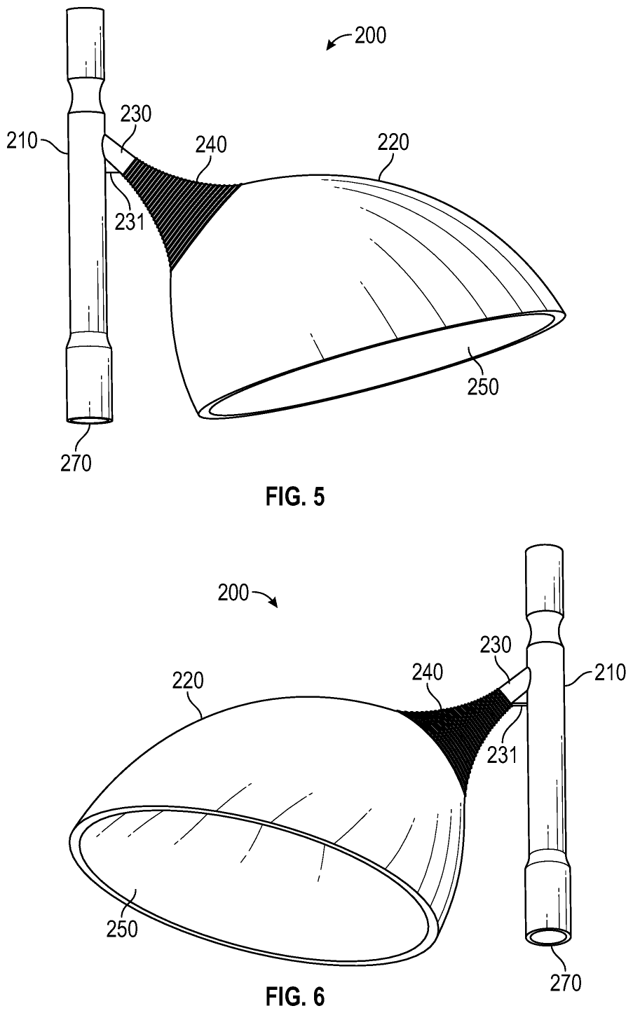

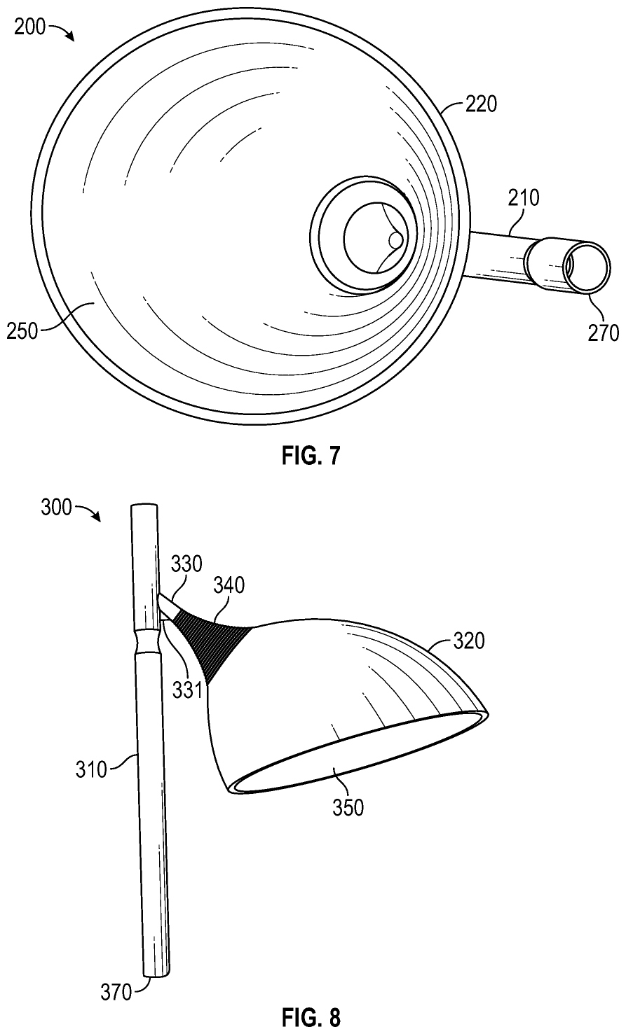

FIGS. 5-7 show another illustration of the suction device according to an embodiment of the present disclosure.

Referring to FIGS. 5-7, a suction device 200 according to an embodiment of the present invention may include a tube 210 connected to a cup 220 via a connector 230 at one end and a suction motor at the opposite end 270. In some embodiments, the cup 220 can be rigidly connected (e.g., not being able to move or pivot) to the tube 210 via the connector 230. In some embodiments, the connector 230 may include additional support portions 231 to help maintain a sturdy connection between the cup 220 and the tube 210. The additional support portions 231 may include rigid materials that extends from the tube 210 to the connector 230 or from the tube 210 to a base of the cup 220. In some embodiments, the cup 220 can include a flexible base 240 that is positioned adjacent to the connector 230. The flexible base 240 can include flexible material that can allow the cup 220 to be adjusted closer or further from the oral and nasal cavities of a person. Additionally, the flexible base 240 can allow the angle of the opening to be adjusted to different angles.

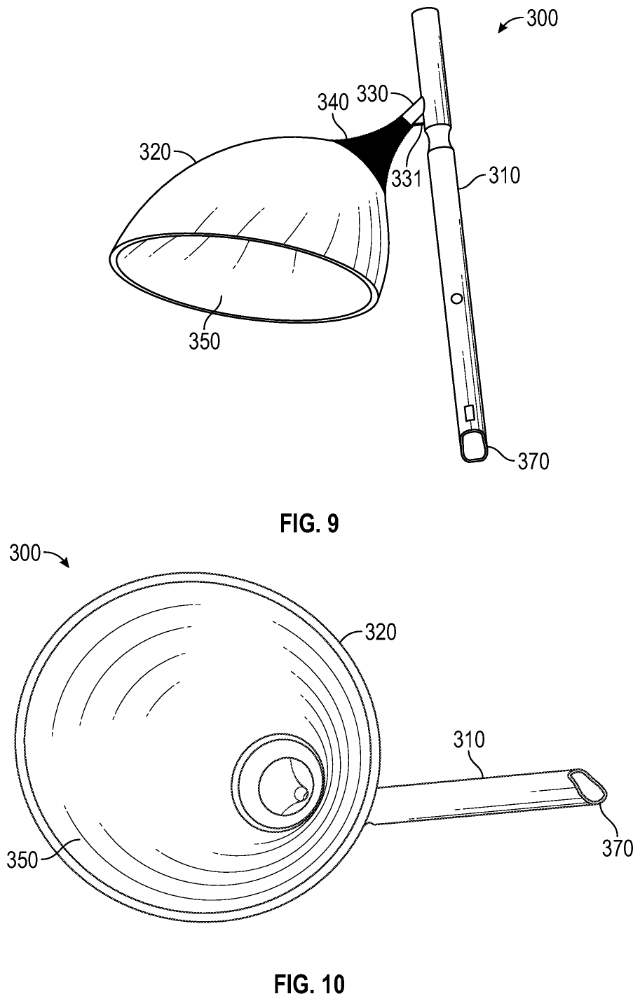

FIGS. 8-10 show another illustration of the suction device according to an embodiment of the present disclosure.

Referring to FIGS. 8-10, a suction device 300 according to an embodiment of the present invention may include a tube 310 connected to a cup 320 via a connector 330 at one end and a suction motor at the opposite end 370. In some embodiments, the cup 320 can be rigidly connected (e.g., not being able to move or pivot) to the tube 310 via the connector 330. In some embodiments, the connector 330 may include additional support portions 331 to help maintain a sturdy connection between the cup 320 and the tube 310. The additional support portions 331 may include rigid materials that extends from the tube 310 to the connector 330 or from the tube 310 to a base of the cup 320. In some embodiments, the cup 320 can include a flexible base 340 that is positioned adjacent to the connector 330. The flexible base 340 can include flexible material that can allow the cup 320 to be adjusted closer or further from the oral and nasal cavities of a person. Additionally, the flexible base 340 can allow the angle of the opening to be adjusted to different angles.

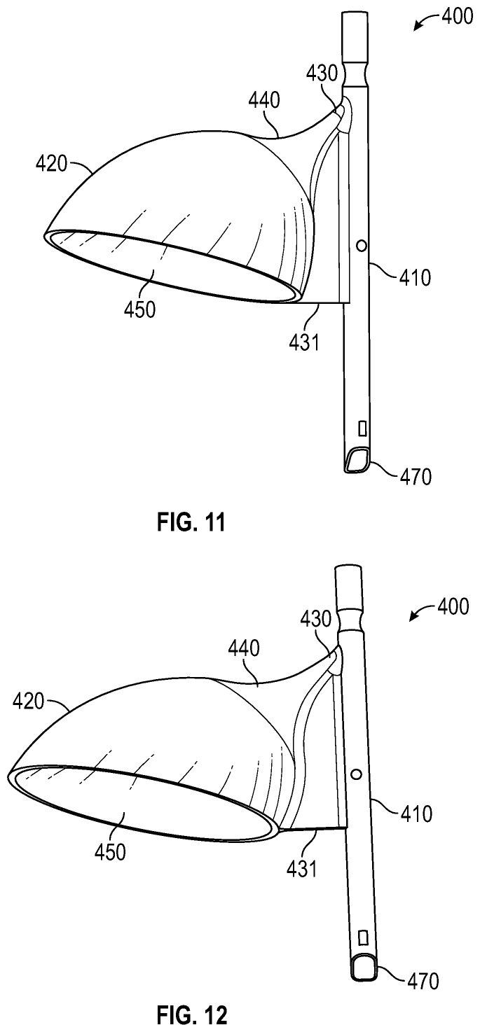

FIGS. 11 and 12 show another illustration of the suction device according to an embodiment of the present disclosure.

Referring to FIGS. 11 and 12 a suction device 400 according to an embodiment of the present invention may include a tube 410 connected to a cup 420 via a connector 330 at one end and a suction motor at the opposite end 470. In some embodiments, the cup 420 can be rigidly connected (e.g., not being able to move or pivot) to the tube 310 via the connector 430. In some embodiments, the connector 430 may include additional support portions 431 to help maintain a sturdy connection between the cup 420 and the tube 410. The additional support portions 431 may include rigid materials that extends from the tube 410 to the connector 430 or from the tube 410 to a base of the cup 420. In some embodiments, the cup 420 can include a rigid base 440 that is positioned adjacent to the connector 430. The rigid base 440 can include rigid material that prevents cup 420 from being moved or pivot at the connector 430. The rigid base 440 may allow for a more sturdy connection between the cup 420 and the tube 410.

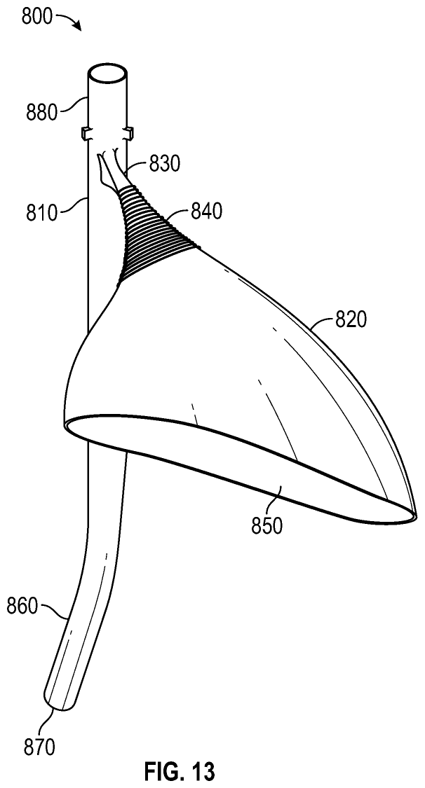

FIG. 13 illustrates a suction device according to an example embodiment of the present disclosure.

Referring to FIG. 13, the suction device 800 can include a tube 810 connected to a cup 820 via a connector 830 at one end and a suction motor at the opposite end 870. In some embodiments, the tube 810 can include an angle 860. The angle 860 can allow the cup 820 to be positioned over the oral and nasal cavities of a person such that the tube 810 does not come into contact with the person. In some embodiments, the diameter of the tube 810 can be tapered narrowly near end 870 such that the diameter of tube 810 may be slightly more narrow at end 870 than at the opposite end 880.

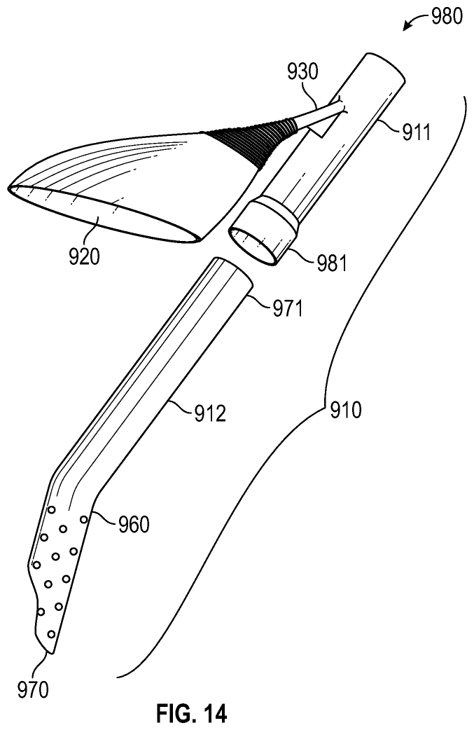

FIG. 14 illustrates two-part suction device according to an example embodiment of the present disclosure.

Referring to FIG. 14, the suction device 900 can include a tube 910 that can include two separate portions: an upper portion 911 and a lower portion 912. The upper portion 911 can be connected to a cup 920 via a connector 930. Further, the upper portion 911 can have an end 980 and an opposite end 981. End 981 can be connected and disconnected from lower portion 912. In some embodiments, end 981 can have a diameter that is wider than the diameter of the lower portion 912. The wider diameter can allow the lower portion 912 to slide into the end 981 to form a connection between the upper portion 911 and the lower portion 912.

The lower portion 912 can include an end 971 and an opposite end 970. The end 971 can connect with the upper portion 911 and the end 970 can connect to the suction motor. Further, the lower portion 912 can include an angle 960. The angle 960 can allow the cup 920 to be positioned over the oral and nasal cavities of a person such that the tube 910 does not come into contact with the person. In some embodiments, the suction motor can be an HVAC suction system used in medical, dental, and veterinary offices.

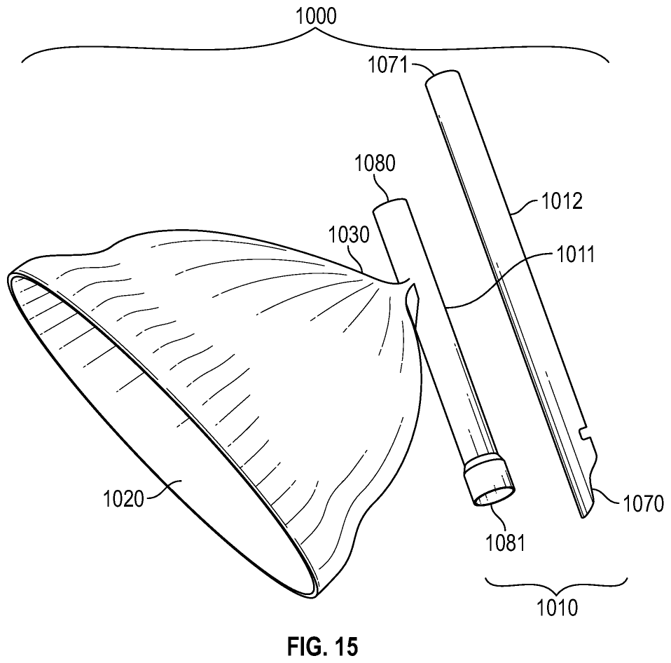

FIG. 15 illustrates a second two-part suction device according to an example embodiment of the present disclosure.

Referring to FIG. 15, the suction device 1000 can include a tube 1010 that may include two separate portions: an upper portion 1011 and a lower portion 1012. The upper portion 1011 can be connected to a cup 1020 via a connector 1030. Further, the upper portion 1011 can have an end 1080 and an opposite end 1081. End 1081 can be connected and disconnected from lower portion 1012. In some embodiments, end 1081 can have a diameter that is wider than the diameter of the lower portion 1012. The wider diameter can allow the lower portion 1012 to slide into the end 1081 to form a connection between the upper portion 1011 and the lower portion 1012.

The lower portion 1012 can include an end 1071 and an opposite end 1070. The end 1071 can connect with the upper portion 1011 and the end 1070 can connect to the suction motor. In some embodiments, the suction motor can be an HVAC suction system used in medical, dental, and veterinary offices.

In the preceding description, for the purposes of explanation, numerous specific details are set forth to provide a thorough understanding of various embodiments. It is apparent, however, that various embodiments may be practiced without these specific details or with one or more equivalent arrangements.

It will be understood that when an element, layer, region, or component is referred to as being “on,” “connected to,” or “coupled to” another element, layer, region, or component, it can be directly on, connected to, or coupled to the other element, layer, region, or component, or one or more intervening elements, layers, regions, or components may be present. However, “directly connected/directly coupled” refers to one component directly connecting or coupling another component without an intermediate component. Meanwhile, other expressions describing relationships between components such as “between,” “immediately between” or “adjacent to” and “directly adjacent to” may be construed similarly. In addition, it will also be understood that when an element or layer is referred to as being “between” two elements or layers, it can be the only element or layer between the two elements or layers, or one or more intervening elements or layers may also be present.

The terminology used herein is for the purpose of describing particular embodiments only and is not intended to be limiting of the present disclosure. As used herein, the singular forms “a” and “an” are intended to include the plural forms as well, unless the context clearly indicates otherwise. It will be further understood that the terms “comprises,” “comprising,” “have,” “having,” “includes,” and “including,” when used in this specification, specify the presence of the stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

As used herein, the term “substantially,” “about,” “approximately,” and similar terms are used as terms of approximation and not as terms of degree, and are intended to account for the inherent deviations in measured or calculated values that would be recognized by those of ordinary skill in the art. “About” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, 5% of the stated value. Further, the use of “may” when describing embodiments of the present disclosure refers to “one or more embodiments of the present disclosure.” As used herein, the terms “use,” “using,” and “used” may be considered synonymous with the terms “utilize,” “utilizing,” and “utilized,” respectively. Also, the term “exemplary” is intended to refer to an example or illustration.

Various embodiments are described herein with reference to sectional illustrations that are schematic illustrations of embodiments and/or intermediate structures. As such, variations from the shapes of the illustrations as a result, for example, of manufacturing techniques and/or tolerances, are to be expected. Further, specific structural or functional descriptions disclosed herein are merely illustrative for the purpose of describing embodiments according to the concept of the present disclosure. Thus, embodiments disclosed herein should not be construed as limited to the particular illustrated shapes of regions, but are to include deviations in shapes that result from, for instance, manufacturing. For example, an implanted region illustrated as a rectangle will, typically, have rounded or curved features and/or a gradient of implant concentration at its edges rather than a binary change from implanted to non-implanted region. Likewise, a buried region formed by implantation may result in some implantation in the region between the buried region and the surface through which the implantation takes place. Thus, the regions illustrated in the drawings are schematic in nature and their shapes are not intended to illustrate the actual shape of a region of a device and are not intended to be limiting. Moreover, the drawings are not necessarily to scale.

Unless otherwise defined, all terms (including technical and scientific terms) used herein have the same meaning as commonly understood by one of ordinary skill in the art to which the present disclosure belongs. It will be further understood that terms, such as those defined in commonly used dictionaries, should be interpreted as having a meaning that is consistent with their meaning in the context of the relevant art and/or the present specification, and should not be interpreted in an idealized or overly formal sense, unless expressly so defined herein.

The foregoing is illustrative of example embodiments, and is not to be construed as limiting thereof. Although a few example embodiments have been described, those skilled in the art will readily appreciate that many modifications are possible in the example embodiments without materially departing from the novel teachings and advantages of example embodiments. Accordingly, all such modifications are intended to be included within the scope of example embodiments as defined in the claims. In the claims, means-plus-function clauses are intended to cover the structures described herein as performing the recited function and not only structural equivalents but also equivalent structures. Therefore, it is to be understood that the foregoing is illustrative of example embodiments and is not to be construed as limited to the specific embodiments disclosed, and that modifications to the disclosed example embodiments, as well as other example embodiments, are intended to be included within the scope of the appended claims. The inventive concept is defined by the following claims, with equivalents of the claims to be included therein.

What is claimed is:

1. A suction device comprising:
a funnel comprising an opening at an end and a base at an opposite end, wherein a diameter of the opening is wider than a diameter of the base;
a tube; and
a suction motor,
wherein a first end of the tube is coupled to the base of the funnel via a connector and a second end of the tube is coupled to the suction motor, and wherein the suction motor is configured to generate a suction force that pulls aerosols from surrounding environment into the opening of the funnel and through the tube and into a collection reservoir for disposal.

2. The suction device of claim 1, wherein the base of the funnel comprises a flexible base.

3. The suction device of claim 1, wherein the suction motor comprises a heating, ventilation, and air conditioning (HVAC) suction system.

4. The suction device of claim 1, wherein the diameter of the opening of the funnel has a range of 4 to 5 inches.

5. The suction device of claim 1, wherein the connector comprises a plurality of support portions that stabilizes the connector.

6. The suction device of claim 1, wherein the tube includes an angled portion.

7. The suction device of claim 1, wherein the base of the funnel comprises a rigid base.

8. A suction device comprising:
a funnel comprising an opening at an end and a base at an opposite end, wherein a diameter of the opening is wider than a diameter of the base;
a tube comprising an upper portion section and a lower portion section, wherein the upper portion section is configured to be coupled and decoupled from the lower portion section; and
a suction motor,
wherein a top end of the upper portion section of the tube is coupled to the base of the funnel via a connector and a bottom end of the lower portion section is coupled to the suction motor, wherein a bottom end of the upper portion section of the tube is coupled to a top end of the lower portion section of the tube, and wherein the suction motor is configured to generate a suction force that pulls aerosols from surrounding environment into the opening of the funnel and through the tube and into a collection reservoir for disposal.

9. The suction device of claim 8, wherein the base of the funnel comprises a flexible base.

10. The suction device of claim 8, wherein the suction motor comprises a heating, ventilation, and air conditioning (HVAC) suction system.

11. The suction device of claim 8, wherein the diameter of the opening of the funnel has a range of 4 to 5 inches.

12. The suction device of claim 8, wherein the connector comprises a plurality of support portions that stabilizes the connector.

13. The suction device of claim 8, wherein the tube includes an angled portion.

14. The suction device of claim 8, wherein the base of the funnel comprises a rigid base.

15. A method for removing aerosols expelled by oral and nasal cavities of a person using a suction device, the method comprising:
coupling a funnel to a first end of a tube via a connector, wherein the funnel comprises an opening at an end and a base at an opposite end, and wherein the base is coupled to the tube via the connector;
coupling a second end of the tube to a suction motor;
positioning the opening of the funnel at a distance from the oral and nasal cavities of the person; and
activating the suction motor, wherein the suction motor is configured to generate a suction force that pulls aerosols from surrounding environment into an opening of the funnel.

16. The method of claim 15, wherein the base of the funnel comprises a flexible base.

17. The method of claim 15, wherein the suction motor comprises a heating, ventilation, and air conditioning (HVAC) suction system.

18. The method of claim 15, wherein the connector comprises a plurality of support portions that stabilizes the connector.

19. The method of claim 15, wherein the tube includes an angled portion.

20. The method of claim 15, wherein the base of the funnel comprises a rigid base.
